(19) United States
(12) Patent Application Publication
(10) Pub. No.: US 2019/0307974 A1
(43) Pub. Date: Oct. 10, 2019

(54) CARTRIDGE, ATOMIZER AND ELECTRONIC CIGARETTE HAVING THE SAME

(71) Applicant: Joyetech Europe Holding GmbH, Zug (CH)

(72) Inventor: Weihua QIU, Changzhou, Jiangsu (CN)

(21) Appl. No.: 16/466,022

(22) PCT Filed: May 8, 2017
(86) PCT No.: PCT/CN2017/083506

§ 371 Date: Jun. 1, 2019

(30) Foreign Application Priority Data
Dec. 2, 2016 (CN) 201611101786.1

(51) Int. Cl. A61M 15/00; A24F 47/00; A61M 11/04

(52) U.S. Cl. CPC A61M 15/0028; A24F 47/008; F17C 9/02; A61M 11/042; A61M 15/002

(57) ABSTRACT

A cartridge for an electronic cigarette includes a cartridge carrier having a liquid storage chamber, an adjusting member, a cartridge base, and an elastic member. The liquid intake member is received in the cartridge carrier and defining a second liquid intake hole fluidly communicating with the liquid storage chamber. The adjusting member has a communicating portion and a sealing portion. The cartridge base is positioned on an end of the cartridge carrier; and the elastic member is received in the cartridge base. An end of the elastic member elastically resists the adjusting member, an opposite end of the elastic member elastically resists the cartridge base to enable the sealing portion to seal the second liquid intake hole.

### FIELD OF TECHNOLOGY

The invention relates to a technical field of smoking simulation, and more particularly, relates to a cartridge, an atomizer having the cartridge, and an electronic cigarette.

### BACKGROUND

Related electronic cigarette generally has a split type structure, the cartridge and the atomizing assembly is connected to each other by a pluggable structure or a simple threaded structure. When the cartridge is detached and separated from the atomizing assembly, the tobacco liquid inlet/outlet of the cartridge remains open, thus the tobacco liquid stored in the cartridge can be leaked out easily. When such kind of electronic cigarette is accessible for children, the cartridge may be opened merely by simple operation such as plugging or rotation, thus children may contact or swallow the tobacco liquid by an accident, it has a potential safety hazard for children's health.

### SUMMARY

Accordingly, it is necessary to provide a cartridge having a children protective function, an atomizer having the cartridge, and an electronic cigarette.

The technical solution adopted by the present disclosure to solve the problem is that:

A cartridge includes a cartridge carrier having a liquid storage chamber, an adjusting member, a cartridge base, and an elastic member. The liquid intake member is received in the cartridge carrier and defining a second liquid intake hole fluidly communicating with the liquid storage chamber. The adjusting member has a communicating portion and a sealing portion. The cartridge base is positioned on an end of the cartridge carrier; and the elastic member is received in the cartridge base. The adjusting member is slidably positioned between the liquid intake member and the cartridge base, an end of the elastic member elastically resists the adjusting member, an opposite end of the elastic member elastically resists the cartridge base to enable the sealing portion to seal the second liquid intake hole, when the adjusting member is moved away from the liquid intake member, the communicating portion is driven to fluidly communicating with the second liquid second liquid intake hole.

Further, an external wall of the adjusting member is provided with at least one guiding protrusion protruding along an axial direction of the adjusting member outwardly, and an internal wall of the cartridge base defines at least one guiding groove along an axial direction of the cartridge base and corresponding to the at least one guiding protrusion, or an external wall of the adjusting member defines at least one guiding groove along an axial direction of the adjusting member, and an internal wall of the cartridge base is provided with at least one guiding protrusion along an axial direction of the cartridge base outwardly and corresponding to the at least one guiding groove, the at least one guiding protrusion is capable of sliding along the at least one guiding groove.

Further, the internal wall of the cartridge base is provided with a resisting portion at an end away from the cartridge carrier, the resisting portion protrudes from a circumferential surface of the cartridge base inwardly, an end of the elastic member elastically resists the resisting portion, an opposite end of the elastic member elastically resists an end of the adjusting member away from the liquid intake member,

Further, the cartridge carrier includes a liquid storage tube and an exhaust tube extending through the liquid storage tube along an axial direction of the liquid storage tube, the liquid storage chamber is formed by a space between an internal wall of the liquid storage tube and an external wall of the exhaust tube, an exhaust passage is formed by an interior space in the exhaust tube.

Further, the cartridge further includes a pressing plate received in the cartridge carrier, wherein the pressing plate is fixedly mounted on an end surface of the liquid intake member away from the adjusting member, the pressing plate defines a first liquid intake hole corresponding to the second liquid intake hole and fluidly communicating with the liquid storage chamber.

An atomizer includes an aforementioned cartridge; an atomizing assembly detachably connected to the cartridge; an atomizing end cover connected to the adjusting member via threads. When the cartridge or the atomizing assembly is rotated, the adjusting member is capable of moving away from or toward the liquid intake member.

Further, the adjusting member is provided with internal threads at an internal wall thereof, the atomizing end cover is provided with external threads at an external wall thereof matching with the internal threads, the external threads of the atomizing end cover extend through the cartridge base and engage the internal threads.

Further, an atomizing chamber is formed by an interior space of the atomizing end cover, the atomizing end cover defines a third liquid intake hole fluidly communicated with the atomizing chamber.

Further, the pressing plate defines a first mounting hole, the liquid intake member defines a second mounting hole, the adjusting member defines a third mounting hole, the atomizing end cover defines a fourth mounting hole, the exhaust tube extends through the first mounting hole, the second mounting hole, the third mounting hole, and the fourth mounting hole, to enable the atomizing chamber to fluidly communicate with the exhaust passage.

An electronic cigarette includes an aforementioned atomizer, wherein the electronic cigarette further includes a battery pack connected to the atomizer.

The advantages of the present disclosure are described as follows:

In the cartridge or electronic cigarette provided by the present disclosure, when in use, the tobacco liquid can flow out the cartridge to be atomized, when the cartridge is disassembled and separated from the atomizing assembly, under the elastic action, the sealing portion of the adjusting member seals the second liquid intake hole subsequently, thus the tobacco liquid is sealed. Even if children contact and open the electronic cigarette, it cannot cause impairment to children, thus it has a better protective function for children, a simple structure and can be easily operated

### BRIEF DESCRIPTION OF THE DRAWINGS

The invention is specifically illustrated with reference to accompanying drawings and embodiments in the following description.

FIG. 1 is a structural view of an electronic cigarette of the preset disclosure;

FIG. 2 is an exploded view of a cartridge of the electronic cigarette of FIG. 1;

FIG. 3 is a structural view of an atomizing assembly and a battery pack of the electronic cigarette of FIG. 1;

FIG. 4 is an exploded view of the electronic cigarette of FIG. 1;

FIG. 5 is a structural view of a cartridge carrier of the electronic cigarette of FIG. 4;

FIG. 6 is a structural view of a liquid intake member of the electronic cigarette of FIG. 4;

FIG. 7 is an enlarged view of an adjusting member of the electronic cigarette of FIG. 4;

FIG. 8 is a structural view of an atomizer base of the electronic cigarette of FIG. 4;

FIG. 9 is an enlarged view of an atomizing end cover of the electronic cigarette of FIG. 4;

FIG. 10 is a cross-sectional view of the electronic cigarette of FIG. 1 (showing a close state of the second liquid intake hole);

FIG. 11 is another cross-sectional view of the electronic cigarette (showing an open state of the second liquid intake hole);

FIG. 12 is another cross-sectional view of the electronic cigarette (rotated for about 90 degrees relative to FIG. 11).

Designations and reference numerals of the part and component in the accompanying drawings.

| | | |
|---|---|---|
| electronic cigarette 100 | cartridge 10 | atomizing assembly 20 |
| cartridge carrier 11 | pressing plate 12 | liquid intake member 13 |
| adjusting member 14 | cartridge base 15 | elastic member 16 |
| housing 21 | battery 22 | atomizing base 23 |
| heating mechanism 24 | atomizing end cover 25 | pressure sensor 26 |
| liquid storage tube 112 | exhaust tube 113 | liquid storage chamber 114 |
| exhaust passage 115 | first mounting hole 121 | first liquid intake hole 122 |
| positioning hole 123 | main body 131 | connecting body 132 |
| third mounting hole 141 | communicating portion 142 | sealing portion 143 |
| guiding protrusion 144 | internal thread 145 | guiding groove 151 |
| resisting portion 152 | first restricting protrusion 153 | air intake hole 211 |
| supporting base 231 | electrode fixing bracket 232 | absorbing member 241 |
| heating member 242 | second restricting protrusion | fourth mounting hole 252 |
| third liquid intake hole 253 | external thread 254 | atomizing chamber 255 |
| resisting portion 1121 | second mounting hole 1311 | second liquid intake hole 1312 |
| positioning protrusion 1313 | first restricting groove 1314 | air intake groove 2311 |
| through hole 2312 | elongated groove 2313 | second restricting groove |

### DETAILED DESCRIPTION OF EMBODIMENTS

The invention is specifically illustrated with reference to accompanying drawings. The accompanying drawings are schematic views which simplified shows fundamental structures of an exemplary embodiment of the invention. Thus, merely the constructions related to the invention are shown.

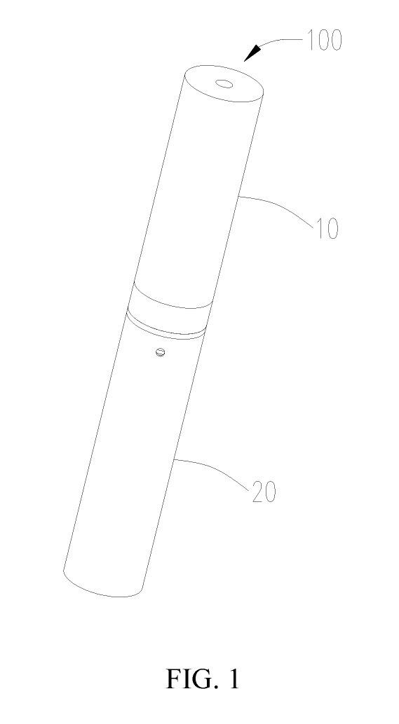

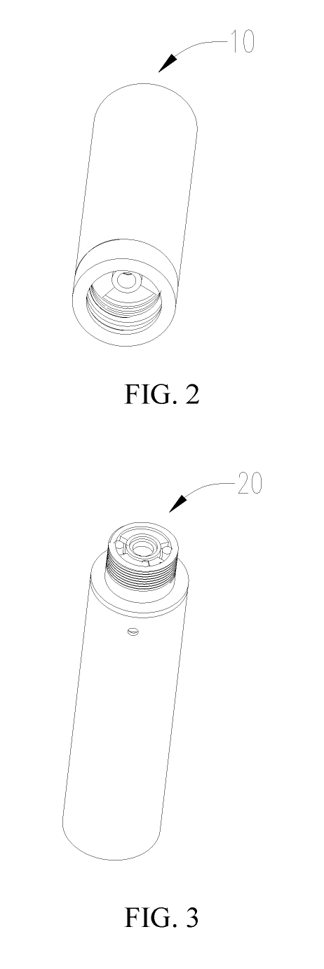

Referring to FIG. 1 through FIG. 3, the present disclosure provides an electronic cigarette 100, the electronic cigarette 100 includes an atomizer (not labeled) and a battery pack (not labeled). The atomizer includes a cartridge 10 configured for storing tobacco liquid, and an atomizing assembly 20 configured for atomizing the tobacco liquid to generate aerosol, the cartridge 10 is detachably connected to the atomizing assembly 20. The battery pack is detachably connected to the atomizing assembly 20 for driven the atomizing assembly 20 to atomize the tobacco liquid.

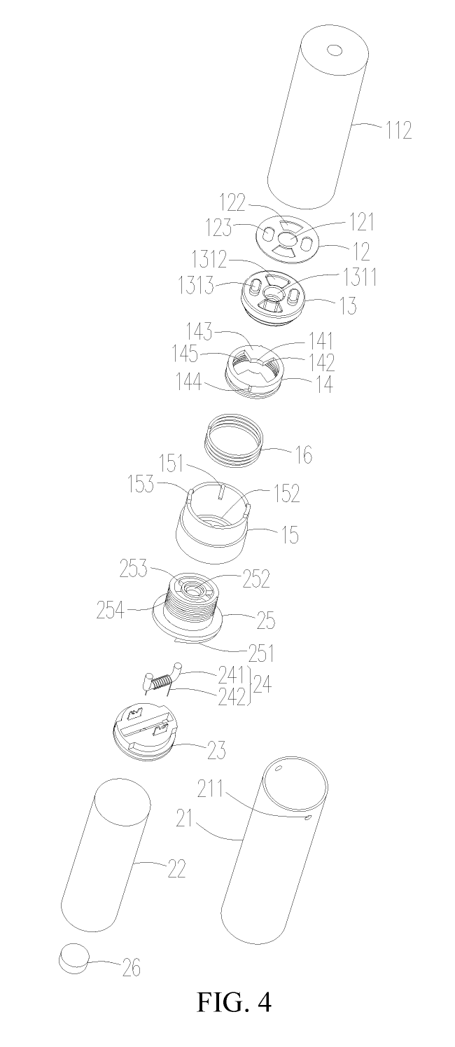

Also referring to FIG. 4, the cartridge 10 includes a cartridge carrier 11, a pressing plate 12, a liquid intake member 13, an adjusting member 14, an elastic member 16, and a cartridge base 15 detachably assembled to an end of the cartridge carrier 11 adjacent to the atomizing assembly 20. The pressing plate 12, the liquid intake member 13, the adjusting member 14, and the elastic member 16 are received in the cartridge carrier 11. The adjusting member 14 is slidably positioned between the liquid intake member 13 and the cartridge base 15. The elastic member 16 is elastically resisted between the adjusting member 14 and the cartridge base 15.

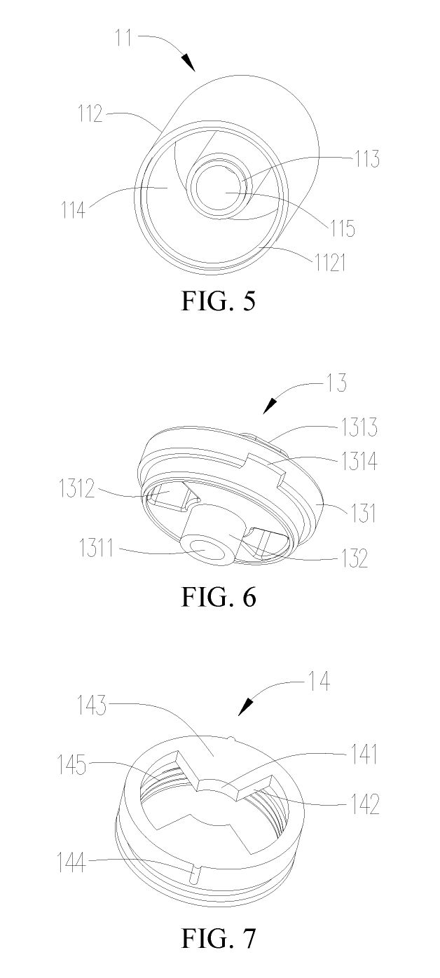

Referring to FIG. 5, the cartridge carrier 11 substantially has a tubular shape. The cartridge carrier 11 includes a liquid storage tube 112 having an opening at an end, and an exhaust tube 113 received in and extending through the liquid storage tube 112. The exhaust tube 113 is positioned on a center of the liquid storage tube 112. A liquid storage chamber 114 is formed between an internal wall of the liquid storage tube 112 and an external wall of the exhaust tube 113, the liquid storage chamber 114 is configured to store the tobacco liquid. The interior space of the exhaust tube 113 forms an exhaust passage 115 for user to inhale. User can smoke via a top of the exhaust tube 113

Further, the liquid storage tube 112 forms a resisting portion 1121 having an annular shape and extending along a circumferential direction of the liquid storage tube 112. The resisting portion 1121 protrudes from the internal wall of the liquid storage tube 112 and is adjacent of the opening.

In the illustrated embodiment, the cartridge carrier 11 can serve as a mouth-piece directly. User keeps an end of the cartridge carrier 11 in his mouth and performs a smoke. It can be understood that, in alternative embodiment, a communicating member can be connected to the cartridge carrier 11 for fluidly communicating with the exhaust tube 113, user keeps an end of the communicating member in his mouth and performs a smoke.

The pressing plate 12 substantially has a disc sheet structure. The pressing plate 12 defines a first mounting hole 121 matching with the exhaust tube 113 at a center of the pressing plate 12. The pressing plate 12 defines a first liquid intake hole 122 and a positioning hole 123. The first liquid intake hole 122 and the positioning hole 123 surround the first mounting hole 121. The pressing plate 12 is sleeved on the exhaust tube 113 via the first mounting hole 121 at the center. The edge of the pressing plate 12 resists the resisting portion 1121 of the liquid storage tube 112, and is connected to the internal wall of the liquid storage tube 112 tightly.

In the illustrated embodiment of the present disclosure, a number of the first liquid intake hole 122 is two, the two first liquid intake holes 122 are symmetrically arranged about the first mounting hole 121. A number of the positioning holes 123 is two, the two positioning holes 123 are symmetrically arranged about the first mounting hole 121. The first liquid intake holes 122 and the positioning holes 123 are uniformly distributed along a circle and are staggered arranged. It can be understood that, in alternative embodiment, the number of the first liquid intake hole 122 and the number of the positioning hole 123 can be one or more than two.

In the illustrated embodiment, the pressing plate 12 is a plastic disc sheet. It can be understood that, in alternative embodiment, the pressing plate 12 can be made of other hard materials such as stainless steel sheet or ceramic sheet.

Referring to FIG. 6, the liquid intake member 13 substantially has a cylinder shape. The liquid intake member 13 includes a main body 131 and a connecting body 132 protruded from a center of the main body 131. The connecting body 132 is positioned on an end of the main body 131 distal from the pressing plate 12. The main body 131 defines a second mounting hole 1311 at the center, the second mounting hole 1311 extends through the connecting body 132 and matches the exhaust tube 113. The main body 131 defines a second liquid intake hole 1312 and forms a positioning protrusion 1313 corresponding to the position hole 123. The second liquid intake hole 1312 is corresponding to the first liquid intake hole 122 and surrounds the second mounting hole 1311 together with the positioning protrusion 1313. The main body 131 further defines a first restricting groove 1314 at a circumferential surface of the main body 131. The liquid intake member 13 is sleeved on an end of the exhaust tube 113 via the second mounting hole 1311. When the positioning protrusion 1313 matches with and is connected to the positioning hole 123, the second liquid intake hole 1312 is fluidly communicated with the first liquid intake hole 122 of the pressing plate 12. The liquid intake member 13 is tightly connected to the internal wall of the liquid storage tube 112. The second mounting hole 1311 is hermetically connected to the external wall of the exhaust tube 113.

In the illustrated embodiment, the liquid intake member 13 is made of rubber. It can be understood that, the liquid intake member 13 can also be made of silica gel, for providing a sealing function

Due to a softness of the material of the liquid intake member 13, the pressing plate 12 closely resists the liquid intake member 13 to enable the liquid intake member 13 not to be deformed, thereby avoiding a liquid leakage.

In the illustrated embodiment, the restricting mode between the liquid intake member 13 and the pressing plate 12 is realized by an engagement between the positioning protrusion 1313 and the positioning hole 123. It can be understood that, the pressing plate 12 can be fixed to the liquid intake member 13 via adhering.

Referring to FIG. 7, the adjusting member 14 substantially has a sleeve shape, and is positioned on an end of the liquid intake member 13 adjacent to the atomizing assembly 20. The adjusting member 14 defines a third mounting hole 141 corresponding to the connecting body 132 at a center of a bottom of the adjusting member 14. The adjusting member 14 defines a communicating portion 142 and is provided with a sealing portion 143 at a bottom of the adjusting member 14. The communicating portion 142 and the sealing portion 143 surround the third mounting hole 141. The adjusting member 14 is sleeved on the connecting body 132 of the liquid intake member 13 via the third mounting hole 141. When the adjusting member 14 closely contacts an end of the liquid intake member 13, the sealing portion 143 can seal the second liquid intake hole 1312 of the liquid intake member 13.

Further, the adjusting member 14 is provided with two guiding protrusions 144 opposite to each other. The two guiding protrusions 144 are protruding along an axial direction of the adjusting member 14 outwardly from an external wall of the adjusting member 14. The guiding protrusion 144 has an elongated shape. The adjusting member 14 is provided with internal threads 145 at an internal wall of the adjusting member 14. It can be understood that, a number of the guiding protrusions 144 can be one or more than two, the guiding protrusion 144 can take on the shape of bumped point.

The cartridge base 15 is substantially a hollow cylindrical structure having two openings at opposite ends. The cartridge base 15 defines two guiding grooves 151 opposite to each other, the two guiding grooves 151 are defined on the internal wall of the cartridge base 15 along an axial direction and corresponding to the two guiding protrusions 144, respectively. The internal wall of the cartridge base 15 is provided with a resisting portion 152 at an end adjacent to the atomizing assembly 20, the resisting portion 152 protrudes from a circumferential surface of the cartridge base 15 inwardly. The cartridge base 15 is provided with a first restricting protrusion 153 at an end away from the atomizing assembly 20. The first restricting protrusion 153 is protruding along an axial direction of the cartridge base 15 and engages the first restricting groove 1314 of the liquid intake member 13.

The cartridge base 15 is fixedly assembled to the opening end of the liquid storage tube 112, the guiding protrusion 144 of the adjusting member 14 is slidable in the guiding groove 151 of the cartridge base 15. A length of the guiding groove 151 along an axial direction of the cartridge base 15 limits a sliding distance of the adjusting member 14.

It can be understood that, the guiding groove 151 can also be defined on the external wall of the adjusting member 14 along an axial of the adjusting member 14. At the time, the guiding protrusion 144 protrudes from the internal wall of the cartridge base 15 outwardly and extends along an axial direction of the cartridge base 15, the guiding protrusion 144 is corresponding to the guiding groove 151, and enables the adjusting member 14 to slide along the guiding groove 151.

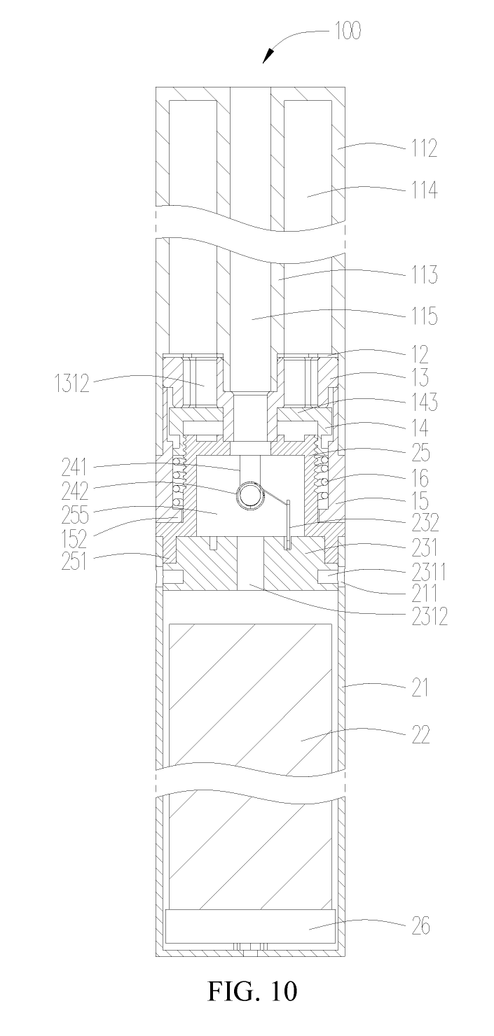

Referring to FIG. 10, an end of the elastic member 16 elastically resists the resisting portion 152, an opposite end of the elastic member 16 resists an end of the adjusting member 14 adjacent to the atomizing assembly 20. At an initial sate, the elastic member 16 tightly presses the adjusting member 14 on the liquid intake member 13, and the sealing portion 143 of the adjusting member 14 seals the second liquid intake hole 1312 of the liquid intake member 13.

In illustrated embodiment, the elastic member 16 is a spring. It can be understood, the form of the elastic member 16 is not limited, the elastic member 16 can be member such as thin steel sheet merely rigidity and elasticity are required, which all fall into the protection scope of the present disclosure.

Referring to FIG. 4, the atomizing assembly 20 includes a housing 21, an atomizing base 23 received in the housing 21, a heating mechanism 24 positioned on the atomizing base 23, and an atomizing end cover 25 detachably positioned on an end of the housing 21.

The battery pack includes a battery 22 received in the housing 21 and a control mechanism (not shown) controlling the battery 22 to direct power for the heating mechanism 24.

The housing 21 substantially has a hollow tubular structure having an opening at an end of the housing 21. The tubular wall of the housing 21 defines two air intake holes 211 opposite to each other. The two air intake holes 211 are adjacent to the opening and are fluidly communicated with an interior of the housing 21. It can be understood that, a number of the air intake hole 211 can also be one or more than two.

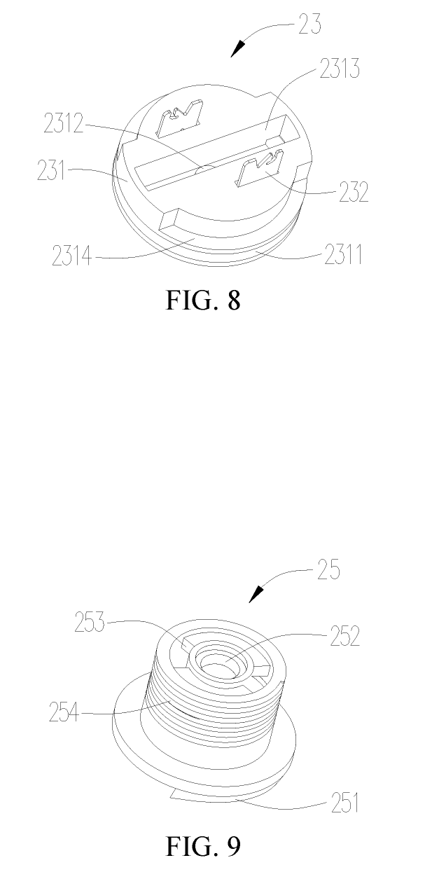

Referring to FIG. 8, the atomizing base 23 has a disc structure, the atomizing base 23 includes a supporting base 231 closely received in the housing 21 and a pair of electrode fixing brackets 232 positioned on the supporting base 231.

The supporting base 231 defines an air intake groove 2311 surrounding a circumferential surface of the supporting base 231 and fluidly communicating with the air intake hole 211. The supporting base 231 defines a through hole 2312 at a lower end of the supporting base 231. The supporting base 231 defines an elongated groove 2313 at an upper end surface and fluidly communicating with the air intake groove 2311 and the through hole 2312. The supporting base 231 further defines a second restricting groove 2314 at an edge of the upper end surface of the supporting base 231.

The electrode fixing bracket 232 extends through the supporting base 231, the pair of electrode fixing brackets 232 are electrically connected to an anode and cathode of the battery 22, respectively.

The heating mechanism 24 includes an absorbing member 241 and a heating member 242 electrically connected to the battery 22. The heating member 242 is wrapped up by the absorbing member 241 or the absorbing member 241 is wrapped up by the heating member 242. The absorbing member 241 is positioned on an upper end surface of the supporting base 231, the absorbing member 241 can be made of materials such as glass fiber or natural fiber which has a better tobacco liquid absorbing performance. Specifically, an end of the heating member 242 is connected to one of the electrode fixing bracket 232, an opposite end of the heating member 242 is connected to the other one electrode fixing bracket 232. The heating member 242 can be made of nichrome wire.

Referring to FIG. 9, the atomizing end cover 25 substantially has a cylindrical structure having opposite ends fluidly communicating with each other and having a purfle. The atomizing end cover 25 is provided with a second latching protrusion 251 corresponding to the second restricting groove 2314, the second latching protrusion 251 protrudes from an end of the atomizing end cover 25 away from the cartridge 10 along an axial direction of the atomizing end cover 25. The atomizing end cover 25 defines a fourth mounting hole 252 at a center of a bottom of an end of the atomizing end cover 25 adjacent to the cartridge 10. The fourth mounting hole 252 is corresponding to the connecting body 132. The atomizing end cover 25 defines a third liquid intake hole 253 surrounding the fourth mounting hole 252. The external wall of the atomizing end cover 25 is provided with external threads 254 engaging with the internal threads 145 of the adjusting member 14.

When assembly, the atomizing end cover 25 latches with the atomizing base 23 via the second latching protrusion 251. An atomizing chamber 255 is constituted by a space formed by the atomizing end cover 25 and the atomizing base 23, the heating mechanism 24 is received in the atomizing chamber 255.

Referring to FIG. 10, in the illustrated embodiment of the present disclosure, the control mechanism includes a pressure sensor 26 positioned in the interior of the housing 21, and a controller (not shown). An end of the controller is connected to the pressure sensor 26, an opposite end of the controller is connected to the battery 22. The pressure sensor 26 is fluidly communicated with the air intake hole 211. When user smokes, a negative pressure is generated in the housing 21, the pressure sensor 26 can detect a signal of a change of the pressure within the housing 21, and transfers the signal to the controller, thereafter, the controller controls the battery 22 to direct power for the heating member 242 of the heating mechanism 24, thereby an automatic powering on and heating can be realized.

It can be understood that, in alternative embodiment of the present disclosure, the control mechanism can also be a switch (not shown) mounted on the external wall of the housing 21, an end of the switch is connected to an electrode of the battery 22, an opposite end of the switch is connected to the electrode fixing bracket 232. The switch can control the on/off state of the battery 22 and the heating mechanism 24. Specifically, the user takes one puff, the switch is required to be toggled one time, the battery 22 provides power of the heating mechanism 24 one time.

The operation procedure of the electronic cigarette 100 of the present discourse is illustrated with reference to accompanying drawings as follow:

When the electronic cigarette 100 of the present disclosure leaves factory, the cartridge 10 and the atomizing assembly 20 have already installed in place, the cartridge 10 and the atomizing assembly 20 are merely required to be aligned and connected to each other, thereafter, the electronic cigarette 100 can be used.

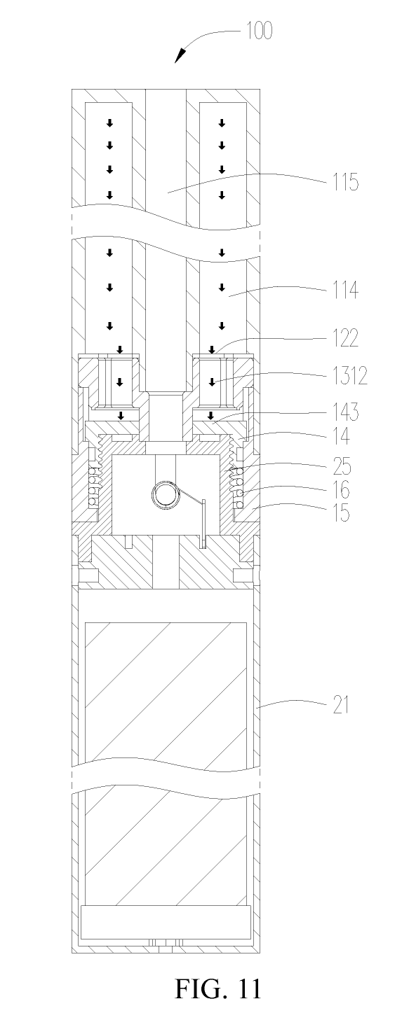

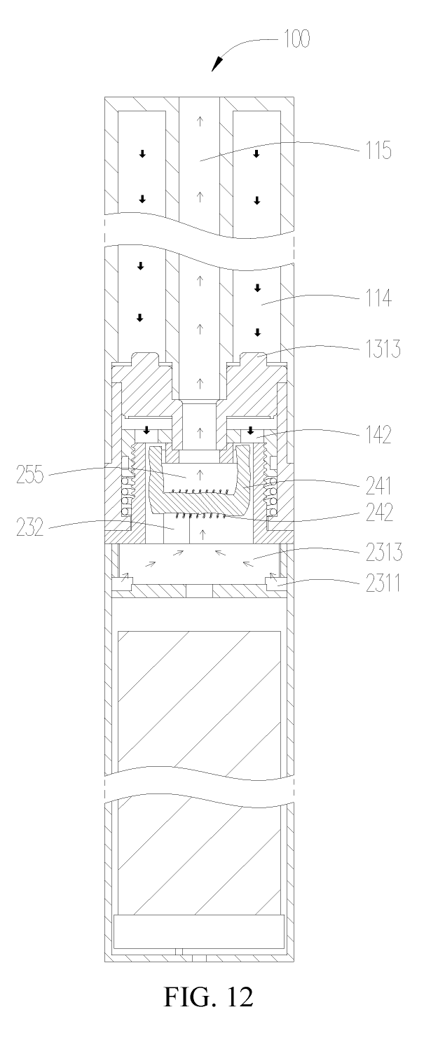

Referring to FIG. 11 and FIG. 12 at the same time, when the cartridge 10 is assembled to the atomizing assembly 20, the cartridge 10 or the atomizing assembly 20 is rotated, a screw force is greater than an elastic force of the elastic member 16, at the time, the external threads 254 of the atomizing end cover 25 engages the internal threads 145 of the adjusting member 14, because the position of the atomizing base 23 remains unchanged, thereby limiting an axial direction of the atomizing end cover 25, causing the adjusting member 14 to slide in the guiding groove 151 of cartridge base 15 and move toward the atomizing end cover 25 along an axial direction of the cartridge base 15, the elastic member 16 is compressed. At the time, the second liquid intake hole 1312 of the liquid intake member 13 is opened by the sealing portion 143 of the adjusting member 14. The tobacco liquid in the liquid storage chamber 114 finally flows into the atomizing chamber 255 of the atomizing end cover 25 by passing through the first liquid intake hole 122, the second liquid intake hole 1312, the communicating portion 142 of the adjusting member 14 and the third liquid intake hole 253, and is then be absorbed by the absorbing member 241, under the heating of the heating member 242, the tobacco liquid is atomized to generate aerosol. The bold arrow in the FIG. 10 and FIG. 11 indicate a flowing direction of the tobacco liquid.

At the same time, the cartridge 10 is fixedly connected to the atomizing assembly 20 via an engagement between the internal threads 145 and the external threads 254, thereby a detachment of the cartridge 10 and the atomizing assembly 20 is avoided.

When user inhales, air enters the electronic cigarette 100 via the air intake hole 211 of the housing 21, and enters the atomizing chamber 2515 via the air intake groove 2311 and the elongated groove 2313 successively. The mixed aerosol passes through the connecting body 132 of the liquid intake member 13 and finally enters user's mouth via the exhaust passage 115. The bold arrow in the FIG. 12 indicate a flowing direction of the air flow.

It can be understood that, the connecting body 132 can be omitted when the exhaust tube 113 is prolonged, to enable the exhaust tube 113 to extend through the pressing plate 12, the liquid intake member 13, the adjusting member 14, and the atomizing end cover 25, causing the exhaust passage 115 to be directly fluidly communicated with the atomizing chamber 2515.

When the cartridge 10 is required to be disassembled, the cartridge 10 or the atomizing assembly 20 is rotated along a reversed direction, by virtue of the engagement between the external threads 254 of the atomizing end cover 25 and the internal threads 145 of the adjusting member 14, the adjusting member 14 moves away from the atomizing end cover 25 along the guiding groove 151 of cartridge base 15, the elastic member 16 returns to an initial position, and compresses the adjusting member 14 on the liquid intake member 13 finally. The sealing portion 143 of the adjusting member 14 seals the second liquid intake hole 1312 of the liquid intake member 13, thereby avoiding a leakage of the tobacco liquid. Even if children contact and open the electronic cigarette, it cannot cause impairment to children.

In the electronic cigarette, when it is assembled and used, the tobacco liquid can flow to be atomized, after being disassembled, the tobacco liquid is sealed, it has a better protective function for children, a simple structure and can be easily operated.

The embodiments described above are merely preferred embodiments, not intended to limit the application. Any modifications, alternatives or improvements made within the principle and spirit of the present application should be interpreted as falling within the protection scope of the present application.

1. A cartridge, comprising:
a cartridge carrier (11) having a liquid storage chamber (114);
a liquid intake member (13) received in the cartridge carrier (11) and defining a second liquid intake hole (1312) fluidly communicating with the liquid storage chamber (114);
an adjusting member (14) having a communicating portion (142) and a sealing portion (143);
a cartridge base (15) positioned on an end of the cartridge carrier (11); and
an elastic member (16) received in the cartridge base (15);
wherein the adjusting member (14) is slidably positioned between the liquid intake member (13) and the cartridge base (15), an end of the elastic member (16) elastically resists the adjusting member (14), an opposite end of the elastic member (16) elastically resists the cartridge base (15) to enable the sealing portion (143) to seal the second liquid intake hole (1312), when the adjusting member (14) is moved away from the liquid intake member (13), the communicating portion (142) is driven to fluidly communicating with the second liquid second liquid intake hole (1312)

2. The cartridge according to claim 1, wherein an external wall of the adjusting member (14) is provided with at least one guiding protrusion (144) protruding along an axial direction of the adjusting member (14) outwardly, and an internal wall of the cartridge base (15) defines at least one guiding groove along an axial direction of the cartridge base (15) and corresponding to the at least one guiding protrusion (144), or an external wall of the adjusting member (14) defines at least one guiding groove (151) along an axial direction of the adjusting member (14), and an internal wall of the cartridge base (15) is provided with at least one guiding protrusion (144) along an axial direction of the cartridge base (15) outwardly and corresponding to the at least one guiding groove (151), the at least one guiding protrusion (144) is capable of sliding along the at least one guiding groove (151).

3. The cartridge according to claim 2, wherein the internal wall of the cartridge base (15) is provided with a resisting portion (152) at an end away from the cartridge carrier (11), the resisting portion (152) protrudes from a circumferential surface of the cartridge base (15) inwardly, an end of the elastic member (16) elastically resists the resisting portion (152), an opposite end of the elastic member (16) elastically resists an end of the adjusting member (14) away from the liquid intake member (13),

4. The cartridge according to claim 1, wherein the cartridge carrier (11) comprises a liquid storage tube (112) and an exhaust tube (113) extending through the liquid storage tube (112) along an axial direction of the liquid storage tube (112), the liquid storage chamber (114) is formed by a space between an internal wall of the liquid storage tube (112) and an external wall of the exhaust tube (113), an exhaust passage (115) is formed by an interior space in the exhaust tube (113).

5. The cartridge according to claim 4, further comprising a pressing plate (12) received in the cartridge carrier (11), wherein the pressing plate (12) is fixedly mounted on an end surface of the liquid intake member (13) away from the adjusting member (14), the pressing plate (12) defines a first liquid intake hole (122) corresponding to the second liquid intake hole (1312) and fluidly communicating with the liquid storage chamber (114)

6. An atomizer, comprising:
a cartridge (10) according to claim 1;
an atomizing assembly (20) detachably connected to the cartridge (10);
an atomizing end cover (25) connected to the adjusting member (14) via threads;
wherein when the cartridge (10) or the atomizing assembly (20) is rotated, the adjusting member (14) is capable of moving away from or toward the liquid intake member (13).

7. The atomizer according to claim 6, wherein the adjusting member (14) is provided with internal threads (145) at an internal wall thereof, the atomizing end cover (25) is provided with external threads (254) at an external wall thereof matching with the internal threads (145), the external threads (254) of the atomizing end cover (25) extend through the cartridge base (15) and engage the internal threads (145).

8. The atomizer according to claim 6, wherein an atomizing chamber (255) is formed by an interior space of the atomizing end cover (25), the atomizing end cover (25) defines a third liquid intake hole (253) fluidly communicated with the atomizing chamber (255).

9. The atomizer according to claim 8, wherein the pressing plate (12) defines a first mounting hole (121), the liquid intake member (13) defines a second mounting hole (1311), the adjusting member (14) defines a third mounting hole (141), the atomizing end cover (25) defines a fourth mounting hole (252), the exhaust tube (113) extends through the first mounting hole (121), the second mounting hole (131), the third mounting hole (1411), and the fourth mounting hole (252), to enable the atomizing chamber (2515) to fluidly communicate with the exhaust passage (115)

10. An electronic cigarette, comprising an atomizer according to claim 6, wherein the electronic cigarette (100) further comprises a battery pack connected to the atomizer.
